(19) United States
(12) Patent Application Publication
(10) Pub. No.: US 2013/0316017 A1
(43) Pub. Date: Nov. 28, 2013

(54) Oral Composition for the Treatment of Acne

(71) Applicant: Michael Hynes, Gibsonia, PA (US)

(72) Inventor: Michael Hynes, Gibsonia, PA (US)

(21) Appl. No.: 13/901,635

(22) Filed: May 24, 2013

Related U.S. Application Data
(60) Provisional application No. 61/651,852, filed on May 25, 2012.

(51) Int. Cl. A61K 36/38; A61K 45/06; A61K 31/12

(52) U.S. Cl. CPC A61K 36/38; A61K 31/12; A61K 45/06
USPC 424/643; 424/769; 424/748; 424/729; 514/679; 424/745

(57) ABSTRACT

Provided is a composition that is useful for treating acne, and which has been found to reduce and/or eliminate acne outbreaks or reduce the severity thereof. The composition includes mangosteen and curcumin. At least a portion of the mangosteen and curcumin can be provided in the form of extracts thereof. The composition can optionally include other components such as guggul, guggul extracts, thyme, thyme extracts, piperine, vitamin B5, vitamin B6, zinc, beta glucans, and green tea extracts. The composition can be provided as an oral administration unit such as a pill or capsule. Also provided is a method of treating acne by ingesting an effective amount of the composition described herein.

### CROSS-REFERENCE TO RELATED APPLICATION

This application claims priority to U.S. Provisional Patent Application No. 61/651,852, filed on May 25, 2012, the entire contents of which are hereby incorporated by reference.

### BACKGROUND OF THE INVENTION

1. Field of the Invention

The invention relates to an oral composition useful in the treatment of acne. More specifically, the invention relates to an antibacterial and anti-inflammatory composition that can reduce or eliminate the number of acne outbreaks and the severity thereof.

2. Description of Related Art

The exact causes of acne are unknown but likely include hormonal changes and the general immunity of an individual as well as said individual's intake of sugar. These causes bring about acne on the body—a result of the skin's oil glands (sebaceous glands) and hair follicles and pores becoming clogged with concomitant lesions, commonly called zits or pimples. Depending on characteristics acne can be broken down into different categories, including:
Comedo—the most basic acne lesion, it is an enlarged and plugged hair follicle;
Whitehead—a comedo that stays fully beneath the skin is called a whitehead;
Blackhead—a comedo that breaches the surface of the skin and opens up is called a blackhead;
Papule—small, tender pink bumps on the skin;
Pustules—also called pimples, these have a pus-filled top and are often also red and highly inflamed;
Cysts—also called nodules, these are large, solid, painful, pus-filled lesions that exist deep within the skin—frequently causing scarring on the skin surface.

The most common method of treating acne is to topically apply creams or ointments directly to skin. Other known methods include administering synthetic, prescription-grade pharmaceuticals, such as those comprising isotretinoin. The drug sold under the tradename Accutane is one such example.

### SUMMARY OF THE INVENTION

Provided is a composition that is useful in preventing the development of or reducing the severity of acne outbreaks. The composition includes mangosteen or mangosteen extracts and curcumin or curcumin extracts, including but not limited to, highly bioavailable extracts thereof. Unlike topical acne products, the composition has been found to reduce acne systemically over the entire body including the face, chest, neck, shoulders and back.

In certain non-limiting embodiments, the composition can include guggul or guggul extracts, piperine, vitamin B5, thyme, vitamin B6, zinc, beta glucans, and/or green tea extract.

The composition can be provided as an oral supplement, such in the form of a pill, capsule or other administration unit, or series of pills or capsules, that can be taken by mouth.

Also provided is a method of treating and/or preventing acne by ingesting an effective amount of the composition described herein.

### DESCRIPTION OF THE PREFERRED EMBODIMENTS

Provided is a composition that is useful for treating acne, and which has been found to reduce and/or eliminate acne outbreaks or reduce the severity thereof. The composition of the present invention includes mangosteen and curcumin. The composition can optionally include other components, as described herein.

Depending on the strength of the composition desired, either mangosteen powder or a mangosteen extract can be used. Unrefined mangosteen powder is useful, but not preferred, because its antioxidant level is relatively low. Mangosteen extracts with high levels of alpha-mangostin and flavonoids can be integrated into the composition to make it a much stronger bactericide as well as being much more effective against inflammation that is endemic to acne.

Curcumin is the principal curcuminoid found in turmeric. However, curcumin as a supplement component is significantly stronger than turmeric. Standard strength curcumin is commonly referred to as “curcumin 95,” meaning that the powder is 95% pure curcumin, a level which is approximately 18-times the amount of curcumin that is found in turmeric. However, therapeutic doses of curcumin for acne can be difficult to achieve with even standard curcumin due to the poor bioavailability caused by the fact that most curcumin, including “curcumin 95” and the minimal curcumin that is naturally found in turmeric, is destroyed within the human body's digestive system within 30 minutes of its being taken. A high level of bioavailable curcumin is preferred for the present composition. Depending on the desired strength of the composition that is being formulated, one can utilize one or more of several approaches to improve bioavailability of the curcumin in the composition.

One such approach is to include piperine in the composition. Piperine, an extract of black pepper, has been found to improve the short-term (0-3 hours) availability of both curcumin and any other compound in the composition.

Another such approach is to include a curcuminoid complex instead of, or in addition to, using “curcumin 95.” A curcuminoid complex combines multiple curcumonoids and volatile oils from turmeric into a more bioavailable form. Such a complex has been found to be absorbed three to four times better than “curcumin 95” and is maintained in blood plasma for a much longer period of approximately 8 hours.

Yet another such approach is to use curcumin that has been encapsulated within phytosomes instead of or in addition to “curcumin 95.” Encapsulating curcuimin in this manner has been found to result in a 20-30× greater bioavailability of curcumin and up to a 50-60× increase in other curcuminoids, including demethoxycurcumin and bisdemethoxycurcumin. High blood plasma levels are also likely maintained for a longer period of time through this method.

Another approach to increasing the bioavailability of curcumin is to use in the composition a solid lipid curcumin particle instead of or in addition to “curcumin 95.” This can result in up to a 65× greater bioavailability of curcumin while likely maintaining blood plasma levels for a longer period of time.

Another such approach is to use a colloidal nano-particle dispersed with or within curcumin instead of or in addition to using “curcumin 95.” This results in up to a 27× greater bioavailability of curcumin while likely maintaining blood plasma levels for a longer period of time.

The above approaches can also be used individually or in any combination thereof to synergistically achieve a level of bioavailability greater than any one method can achieve on its own. The invention further contemplates use of other forms of curcumin, particularly highly bioavailable forms of curcumin, that are available or may become available.

The composition can include other components as well.

For example, the composition can include guggul. Depending on the strength of the composition desired, guggul powder or a guggul extract can be used. Unrefined guggul powder is usable, but not preferred, because its antioxidant level is relatively low. Guggul extracts with high levels of guggulsterones can be integrated into the composition to make it a much stronger bactericide as well as being much more effective against inflammation. Adding a high guggulsterone component to the composition has been found to be particularly effective against severe cystic acne.

The composition can also include thyme. Thyme and thyme extracts have very high antioxidant levels. They have high bactericide and anti-inflammatory effects against acne.

While not wishing to be bound by a single theory, it is believed that the acne reducing/eliminating properties of the present composition work because the components thereof provide bactericidal, anti-inflammatory, and oil-reducing effects.

The antibacterial-providing components of the present invention include mangosteen or mangosteen extract, curcumin, curcumin extract or highly bioavailable curcumin extract, and guggul or high guggulsterone guggul extract. These antibacterial components use natural components to kill bacteria that exist within acne, and particularly within pustules and cysts. In addition, the components also kill bacteria within comedos (whiteheads and blackheads) before the bacteria can multiply and cause a pustule or cyst to be produced. The highly bioavailable abstracts are particularly effective. These extracts have been found to be effective against a wide array of common acne bacteria including propionibacterium acnes, staphylococcus aureus and staphylococcus epidermidis as well as other bacteria that could cause acne.

The anti-inflammatory-providing components include those listed above as providing antibacterial effects. In other words, mangosteen or mangosteen extract, curcumin, curcumin extract or highly bioavailable curcumin extract, guggul or high guggulsterone guggul extract, and thyme and thyme extracts in addition to providing antibacterial effects, also have been found to provide anti-inflammatory effects. The anti-inflammatory action can result in a reduction in the redness, swelling and pain associated with pimples and cysts.

The composition can also include components which reduce oil production. One such component, found to reduce sebum, is vitamin B5. Depending on the amount added to the composition, if at all, sebum production can be lessened to a greater or lesser extent. Reduction in sebum production aids in the treatment of acne because a sebum plug—or clogged pore—is the starting point for most acne. Reducing the amount of sebum that is produced makes it less likely for any acne to occur in the first place.

The compositions described herein have several advantages to synthetically derived pharmaceuticals, such as isotretinoin-based compositions. For one, the inventive composition is comprised of an assemblage of naturally derived plant extracts. In addition, synthetic acne products can be made to extraordinarily high potency—so high that the total daily dose required can be low, such as between 40-120 mg per day. The difficulty in utilizing naturally derived plants is in assimilating sufficiently high doses of critical ingredients to be effective. This limitation is made more difficult because some components—such as curcumin—have very poor innate bioavailability within the human body. Utilizing the techniques outlined above, an efficacious effect can be achieved with mangosteen and curcumin, and optionally other components such as guggul.

The effective dosage of the composition of the present invention is typically between about 800 and 5,400 mg per day, such as between 1,700 and 3,400 mg per day. The composition typically can include between 10% and 70% mangosteen, such as between 30% and 55%, between 7% and 50% curcumin, such as between 13% and 26%, between 0% and 25% guggul, such as between 5% and 13%, and between 0% and 50% of other components, such as between 0% and 35%, such as thyme and vitamin B5. The effective dosage is dependent upon the physiology of the individual as well as the particular strength of the components which make up the composition. For example, a composition which uses high-xanthone mangosteen extract would have a lower effective dosage than a composition which uses mangosteen powder or a lower-xanthone mangosteen extract. The same general principle applies to the other components.

Also provided is a method of treating and/or preventing acne. The method includes administering an effective dosage of the above composition, preferably split over a twice-daily administration. The composition can be provided in various dosage forms, such as a pill, tablet, or capsule, with each individual administration unit (e.g, each pill, capsule, tablet, etc.) comprising a certain dosage of the composition, which dosage may be within the effective dosage or may be more or less than the effective dosage, though preferably each unit contains a dosage that is within the effective dosage range or less than the effective dosage range, allowing more than one unit to be taken to achieve the effective dosage. Alternatively, the composition can be divided into a plurality of pills, capsules, tablets, etc., such that each unit includes one or more of the components. Administration typically involves orally taking and ingesting one or more of the pills.

Also provided is a method of manufacturing a composition for the treatment of acne. The method includes blending together mangosteen and curcumin, and optionally other components such as those described herein. Blending can be done using any suitable mixing device, as would be appreciated by those skilled in the art. The method can further include encapsulating the composition or otherwise forming the composition into a form which can be administered orally, including as a liquid or as a pill, capsule, or other administration unit as described above.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements. For example, it is to be understood that the present invention contemplates that, to the extent possible, one or more features of any embodiment can be combined with one or more features of any other embodiment.

What is claimed is:

1. A composition for the treatment of acne, comprising mangosteen and curcumin.

2. The composition of claim 1, wherein at least a portion of the mangosteen is provided as a mangosteen extract and at least a portion of the curcumin is provided as a curcumin extract.

3. The composition of claim 1, wherein the composition comprises between 10 and 70% mangosteen and between 30 and 55% mangosteen based on the total weight of the composition.

4. The composition of claim 1, wherein at least a portion of the curcumin is provided in a highly bioavailable form.

5. The composition of claim 1, wherein at least a portion of the curcumin is provided as a curcuminoid complex.

6. The composition of claim 1, further comprising one or more of guggul, guggul extracts, thyme, thyme extracts, piperine, vitamin B5, vitamin B6, zinc, beta glucans, and green tea extracts.

7. The composition of claim 2, further comprising piperine.

8. The composition of claim 2, further comprising guggul or extracts thereof.

9. A method of treating acne, comprising:
ingesting, on a periodic basis and over a time sufficient to treat the acne, an effective amount of a composition comprising mangosteen and curcumin.

10. The method of claim 9, wherein the effective amount is between 800 and 5,400 mg per day.

11. The method of claim 9, wherein at least a portion of the mangosteen is provided as a mangosteen extract and at least a portion of the curcumin is provided as a curcumin extract.

12. The method of claim 11, wherein the composition further comprises one or more of guggul, guggul extracts, thyme, thyme extracts, piperine, vitamin B5, vitamin B6, zinc, beta glucans, and green tea extracts.

13. The method of claim 9, wherein the composition is provided in the form of one or more administration units that are administered orally.

14. The method of claim 13, wherein a plurality of administration units are administered orally, each of which comprises mangosteen, curcumin, or guggul.

15. An administration unit designed to be administered orally for the treatment of acne, comprising mangosteen and curcumin.

16. The administration unit of claim 15, wherein at least a portion of the mangosteen is provided as a mangosteen extract and at least a portion of the curcumin is provided as a curcumin extract.

17. The administration unit of claim 16, further comprising one or more of guggul, guggul extracts, thyme, thyme extracts, piperine, vitamin B5, vitamin B6, zinc, beta glucans, and green tea extracts.

18. The administration unit of claim 17, comprising between 10 and 70% mangosteen and between 30 and 55% mangosteen based on the total weight of the unit.

19. A system for the treatment of acne, comprising a plurality of administration units which each comprise mangosteen, curcumin, or guggul.

20. The system of claim 19, wherein at least a portion of the administration units comprise two or more of mangosteen, curcumin, and guggul.
